(19) United States
(12) Patent Application Publication
(10) Pub. No.: US 2003/0079741 A1
(43) Pub. Date: May 1, 2003

(54) Breathing aid

(76) Inventor: Craig Sawka, Grosse Ile, MI (US)

Correspondence Address: Richard D. Grauer, Rader, Fishman & Grauer PLLC, Suite 140, 39533 Woodward, Bloomfield Hills, MI, 48304, US

(21) Appl. No.: 10/015,182

(22) Filed: Oct. 30, 2001

(51) Int. Cl. A61M 16/00

(52) U.S. Cl. 128/200.11; 128/204.14

(57) ABSTRACT

A breathing aid device is disclosed which includes an air stream source, a mouthpiece that is received in the mouth of a user, a conduit interconnecting the air stream source and the mouthpiece, and a water source that introduces water into the air stream, where liquid water droplets are periodically sprayed through the mouthpiece and into the mouth of a user by the air stream. A method of relieving parched breathing passages is also disclosed, which involves providing a mouthpiece connected to an air stream, periodically spraying liquid water droplets, carried by said air stream, through said mouthpiece into the mouth of the user.

### FIELD OF THE INVENTION

This invention relates to the prevention of parching of the mouth of a user with blocked nasal passages.

### BACKGROUND OF THE INVENTION

A common problem among medical patients is the drying or parching of the breathing passages including the nasal passages, the mouth and throat. Parching is particularly acute when the nasal passages of the user are blocked due to injury, disease or as a consequence of surgery. Blocked nasal passages may make a user generally uncomfortable because they must breathe through their open mouth. Breathing through an open mouth, in a matter of minutes, can lead to a parched mouth and throat. This is true whether the user is trying to sleep or merely rest comfortably.

During sleep, a parched mouth can be so uncomfortable that the person wakes to purposefully moisten the mouth and throat. Further, it is often difficult for a person to maintain an open mouth during sleep, thus causing repeated waking to re-open the mouth. The resulting sleep deprivation is similar to the repeated waking associated with sleep apnea due to stoppages in breathing.

It is known to artificially humidify air that is to be inhaled. One known device for preventing parching of the mouth is a re-breather, comprising a mouthpiece-attached chamber containing a plurality of protuberances. As the user exhales, water in the exhaled air condenses on the protuberances. Upon inhalation, the ambient air is drawn over the protuberances and is humidified to some degree. However, the protuberances cannot sufficiently moisten the ambient air to a level which would prevent parching. Indeed, parching is still a problem when the relative humidity of the ambient air is high. More moisture is required to prevent parching than can be provided by the water condensed from the exhaled air.

The inventor has created an improved method and device for preventing mouth and throat parching for users with blocked nasal passages.

### SUMMARY OF THE INVENTION

The present invention is a breathing aid with an air stream source, a mouthpiece received in the mouth of a user, a conduit interconnecting the air stream source and the mouthpiece, and a water source that introduces water into the air stream, where liquid water droplets are periodically sprayed through the mouthpiece and into the mouth of a user by the air stream.

The present invention is also a method of relieving parched breathing passages that includes periodically spraying liquid water droplets through a mouthpiece retained in the mouth of the user.

### BRIEF DESCRIPTION OF THE DRAWINGS

In the drawings:

FIG. 1 shows an exploded view of the mouthpiece and nozzle and conduit combination.

FIG. 2 shows an embodiment utilizing humidified air.

FIG. 3 shows an embodiment utilizing liquid water.

### DETAILED DESCRIPTION

The present invention includes a mouthpiece placed in the user's mouth prior to prevent parching of the mouth and throat. The mouthpiece is adapted to receive a conduit that conducts air and droplets of water into the user's mouth. While especially intended for people with blocked nasal passages, the present invention can be utilized by anyone that has difficulty with parching of the mouth and throat.

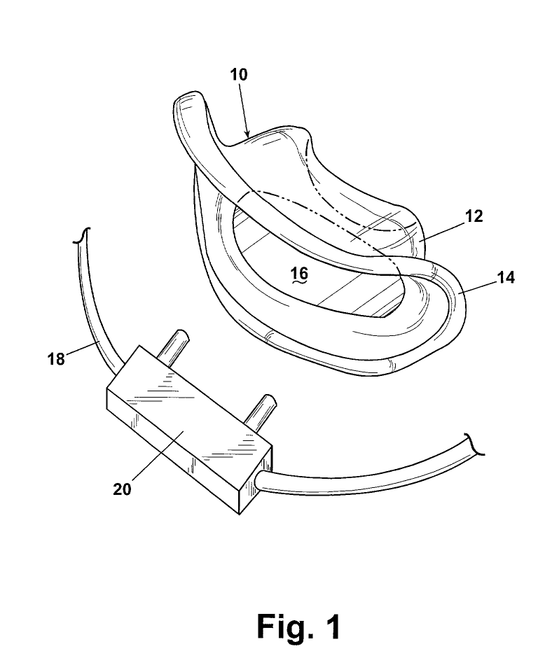

In a useful embodiment, as shown in FIG. 1, mouthpiece 10 has an interior portion 12 that holds the user's lips and teeth slightly apart. An exterior portion 14 of the mouthpiece helps keep the mouthpiece from falling into the user's mouth. The mouthpiece also has a passageway 16 through which the user breathes and through which water is sprayed into the user's mouth. The preferred structure for interior portion 12 is a tube that defines the circumference of passageway 16. However, other structures such as posts that space the teeth and lips are also contemplated. Exterior portion 14 is preferably a flange that is generally perpendicular to the passageway 16. However, the exterior portion may be any structure or device that keeps the mouthpiece from falling into or out of the user's mouth, including a tether affixed behind the user's ears or head.

The conduit may be made of any conventional tubing. One end of the conduit is connected to the mouthpiece, while the other end is connected to the source of water and/or air. The conduit is received in the mouthpiece passageway to convey water and air into the user's mouth. A nozzle may be used to facilitate the formation of a spray of water droplets. One particularly useful conduit 18 and nozzle 20 combination is a conventional nasal cannula, as shown in FIG. 1. Nasal cannulae are commonly found in health care facilities and carried by medical equipment suppliers, and thus would require negligible start-up costs for use in the present invention. In addition, nasal cannulae are easily secured in placed on the user so as to prevent dislodging of the conduit or the mouthpiece. In another useful embodiment, the conduit and mouthpiece are integral to one another.

In operation, the breathing aid produces a periodic spray of water droplets into the mouth of the user. It is contemplated that everything less than a continuous spray of water will constitute a periodic spray. The number of sprays in a given time period, i.e., the rate, and the volume of water in each spray is selectable by the user. Generally, the rate of respiration and the ambient humidity will help determine the rate and volume of water required by the user. The volume of the sprayed water will typically be less then 10 mL and preferably the volume will be about 1 mL. The rate at which the water is sprayed will typically be one spray every 1 to 2 minutes but may be as low as one spray every sixty minutes and as high as once every 2 to 3 seconds. In any case, during the use of the breathing aid by a user, there is, whether regular or irregular, a pattern of periodic water sprays added to the air flowing through the mouthpiece.

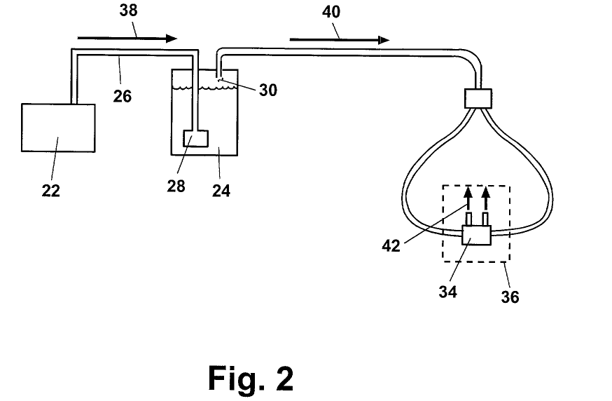

The spray of water droplets can be produced by at least two different methods. In both preferred methods, a stream of air in conducted through a conduit to the user's mouth. The first device, shown in FIG. 2, comprises an air stream source 22 connected to water source 24 via first conduit 26. Diffuser 28 is preferably connected to the end of conduit 26 lodged in water source 24 below the water level. Water source 24 has outlet 30 to which second conduit 32 is attached. Conduit 32 includes a nozzle 34 adapted to be received in mouthpiece 36 (shown schematically).

Air stream source 22 preferably provides a continuous flow of air (shown by arrow 38) to water source 24, where the air is humidified by the water, preferably to the point of saturation. The humidified air flow (shown by arrow 40) is forced out outlet 30 and through conduit 32. Significantly, while in conduit 32, a portion of the water in the humidified air condenses and collects as liquid.

Eventually, the condensed water will substantially block conduit 32, restricting the air flow sufficiently to cause an air pressure increase behind the blockage. The pressure increases until it overcomes the blockage and sprays water droplets and air (shown by arrows 42) into the user's mouth. No misting nozzle is required, although one can be used, in this embodiment because the intermittent spraying of water by the air provides the desired water droplets to comfortably moisten the user's mouth and throat. To increase the amount of water which condenses out of the humidified air in the second conduit, the water source is preferably heated. Although preferably not used, a cooling bath may also be placed around the second conduit to facilitate condensation.

The volume of water sprayed is related to the length and internal diameter of the second conduit. As the length and internal diameter increases, so does the volume. The rate of water spraying is related to the difference in temperature of the humidified air as it leaves the outlet and the ambient temperature along the second conduit. The larger the difference in temperature, the more water that will condense in the second conduit, thus increasing the rate at which water is sprayed into the mouth of the user. In addition, relative humidity of the air as it leaves the water source will also affect the rate of spraying. The closer to 100% relative humidity, the more water that will condense over a given decrease in temperature. Further, the flow rate of the air stream will relate to the rate of spraying.

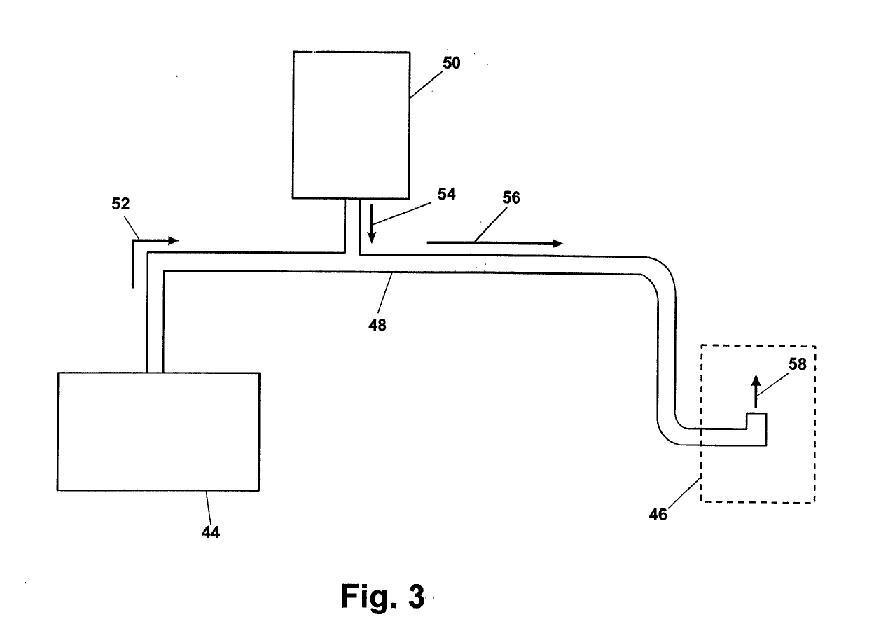

The second device for providing a water spray is shown in FIG. 3. Air stream source 44 is connected to mouthpiece 46 (shown schematically) via conduit 48. Water source 50 introduces water into conduit 48 in a continuous stream or in a discrete droplet form.

Air stream source 44 preferably provides a continuous flow of air (shown by arrow 52) into which water source 50 introduces water (shown by arrow 54). The air forces the water (shown by arrow 56) down conduit 48 and eventually will spray the water and air (shown by arrow 58) out of mouthpiece 46 into the user's mouth. No misting nozzle is required, although one can be used, in this embodiment because the spray of water and air provides any desired misting of water to comfortably moisten the user's mouth and throat. As one skilled in the art will appreciate, the volume and rate of water spray provided by this method may be easily selected by the user (through pump or valve controls, not shown)and is determined by a combination of the flow rate of the air and the rate of introduction of the water.

A user using the present invention during sleep will benefit from a reduction in the episodic waking related to a parched mouth and throat. A concordant reduction in sleep deprivation should also result from the use of this invention because restful sleep will now be possible for people with parched mouths and throats.

While the invention has been specifically described in connection with certain specific embodiments thereof, it is to be understood that this is by way of illustration and not of limitation, and the scope of the appended claims should be construed as broadly as the prior art will permit.

What is claimed is:

1. A breathing aid comprising:
an air stream source;
a mouthpiece adapted to be received in the mouth of a user;
a conduit interconnecting said air stream source and said mouthpiece for supplying an air stream to said mouthpiece in a manner such that said air stream will be inhaled by a user of said mouthpiece; and
a water source adapted to introduce water into said air stream,
wherein water droplets are periodically sprayed through said mouthpiece and into the mouth of a user by said air stream.

2. The breathing aid of claim 1, wherein said water droplets result from liquid water injected into said air stream from said water source.

3. The breathing aid of claim 1, wherein said water droplets result from water condensed from said air stream.

4. The breathing aid of claim 3, wherein said water source is adapted to humidify said air stream.

5. The breathing aid of claim 4, further including a condensing means between said water source and said mouthpiece for causing partial condensation of water from said humidified air stream.

6. The breathing aid of claim 5, wherein said condensing means includes a selection of operating parameters, including the temperature of said humidified air stream relative to ambient air, the length and internal diameter of said conduit, and the flow rate of said air stream, wherein said condensed water accumulates in said conduit and partially obstruct the air stream until the resulting air pressure increase in the conduit causes the water droplets to be sprayed through the mouthpiece and into the mouth of a user.

7. The breathing aid of claim 4, wherein partial condensation of water from said humidified air stream occurs between said water source and said mouthpiece.

8. The breathing aid of claim 7, wherein said condensed water accumulates in said conduit until said air stream causes the water droplets to be sprayed through mouthpiece and into the mouth of a user.

9. The breathing aid of claim 8, wherein operating parameters, chosen from temperature of said humidified air stream relative to ambient temperature, relative humidity of said humidified air stream relative to ambient relative humidity, length and internal diameter of said conduit flow rate of said air stream or combinations thereof, are manipulated to provide a desired rate of spraying said water droplets through said mouthpiece and into the mouthpiece of the user.

10. A method of relieving parched breathing passages, comprising:
providing a mouthpiece having a fluid passageway fluidly connected to an air stream source; and
periodically spraying water droplets, carried by said air stream, through said mouthpiece passageway.

11. The method of claim 10, further including, prior to said spraying step, introducing water into said air stream.

12. The method of claim 11, wherein said introducing step includes injecting liquid water into said air stream.

13. The method of claim 11, wherein said introducing step includes humidifying said air stream.

14. The method of claim 13, further including, after said introducing step and prior to said spraying step, condensing liquid water from said humidified air stream.

15. The method of claim 14, wherein said spraying step results from said liquid water accumulating and partially obstructing the air stream until the resulting air pressure rise causes said liquid water to be sprayed through said mouthpiece passageway.

16. A breathing aid comprising:
an air stream source;
a mouthpiece adapted to be received in the mouth of a user;
a conduit interconnecting said air stream source and said mouthpiece for supplying an air stream to said mouthpiece in a manner such that said air stream will be inhaled by a user of said mouthpiece; and
a water source adapted to humidify said air stream,
wherein partial condensation of water from said humidified air stream occurs between said water source and said mouthpiece and water droplets are periodically sprayed through said mouthpiece by said air stream.

17. The breathing aid of claim 16, further including a condensing means between said water source and said mouthpiece for causing partial condensation of liquid water from said humidified air stream.

18. The breathing aid of claim 17, wherein said condensing means includes a selection of operating parameters, including the temperature of said humidified air stream relative to ambient air, the length and internal diameter of said conduit, and the flow rate of said air stream, whereby said liquid water droplets accumulate in said conduit and partially obstruct the air stream until the resulting air pressure rise in the conduit causes the water droplets to be sprayed through the mouthpiece.
